(19) United States
(12) Patent Application Publication
(10) Pub. No.: US 2003/0018085 A1
(43) Pub. Date: Jan. 23, 2003

(54) Isostearic acid salts as permeation enhancers

(76) Inventors: Araz A. Raoof, Dublin (IE); Mangaraju Gudipati, Yardley, PA (US)

Correspondence Address: SYNNESTVEDT & LECHNER, LLP, 2600 ARAMARK TOWER, 1101 MARKET STREET, PHILADELPHIA, PA, 191072950

(21) Appl. No.: 10/145,180

(22) Filed: May 13, 2002

Related U.S. Application Data
(60) Provisional application No. 60/290,437, filed on May 11, 2001.

(51) Int. Cl. A61K 47/00

(52) U.S. Cl. 514/772; 514/784

(57) ABSTRACT

A pharmaceutical composition comprising a drug and a permeation enhancer that comprises a mixture of compounds, said mixture containing a major amount of compound having a multi-carbon backbone having a partially or completely neutralized acid functional group and also one or more side chains which have one or more carbon atoms and, optionally, one or more functional groups.

This application is related to and claims the of priority to U.S. Provisional Application No. 60/290,437, filed May 11, 2001.

### FIELD OF THE INVENTION

The present invention relates to permeation enhancers that are useful in the administration of a drug.

Drug delivery systems generally involve a permeation step followed by absorption into the circulatory system. For example, a drug can be applied through the skin by use of a transdermal patch which comprises a drug and a film or fabric and which is adhered to the outer skin of the patient. Drugs are delivered also across a mucous membrane or other cellular membrane (collectively “transmucosal”), for example, by: (A) aerosol delivery of the drug to the nose or lungs; (B) oral ingestion of the drug followed by permeation through the gastrointestinal wall; and (C) the dissolution of lozenges or pills held between the cheek and gum or under the tongue followed by transport through the membranes of the mouth.

During the early development of transdermal delivery systems, investigators found that the oily, hydrophobic nature of the skin reduces significantly the absorption rate of aqueous drug solutions or dispersions. Thus, the natural barrier properties of skin, which protect the body against the ingress of foreign substances, act also as barriers to applied drugs, thereby reducing their rate of permeation and ultimately their bioavailability. Problems are encountered also in delivering drugs in a satisfactory way by transmucosal means. The rate of drug permeation is an important factor in achieving bioavailability and pharmaceutically useful concentrations of the drug at the target membrane. It is not surprising that considerable effort has been dedicated toward the objective of enhancing the rate of drug permeation through the skin or by transmucosal means. Examples of such efforts are summarized below.

### REPORTED DEVELOPMENTS

U.S. Pat. No. 5,854,281 (Uekama, et al.) teaches the use of straight chain fatty acids, salts, and esters thereof to enhance the percutaneous permeability of prostaglandin. U.S. Pat. Nos. 5,952,000 and 5,912,009 (Venkateshwaran, et al.) disclose drug delivery systems that are enhanced by the presence of a fatty acid ester of lactic acid (or salts thereof) and a fatty acid ester (or salts thereof) of glycolic acid respectively. The use of glycerides of fatty acids to enhance the skin permeation of a biologically active pergolide is disclosed in U.S. Pat. No. 6,001,390 (Yum, et al.). U.S. Pat. No. 4,789,547 teaches the enhancement of drug permeation through the skin by a saturated or unsaturated fatty acid in a solvent such as propylene glycol. Published PCT application WO00/22909 discloses oral delivery systems for pharmaceutical or other biologically active substances wherein the pharmaceutical or other substance is coated or complexed with a carboxylic acid to enable the substance to transit the stomach and to be absorbed in the intestine. The coating or complexing is achieved by means of co-precipitation from an acidic solution of the active substance and carboxylic acid, which is described as having from nine to 30 carbon atoms in a straight or branched chain, saturated or unsaturated, acyclic or cyclic structure and further substituted or unsubstituted with functional groups such as steroid rings, phenyl groups and the like. WO00/22909 discloses specific examples of complexes formed from the straight chain, saturated or unsaturated or steroidal carboxylic acids, dodecanoic acid, tetradecanoic acid, hexadecanoic acid, octadecanoic acid, oleic acid. palmitoleic acid, ricinoleic acid and fusidic acid.

Investigators continue to seek ways to administer safely and effectively drugs by transmucosal or transdermal routes. Obstacles to these goals are the complexity and variability in the properties of the various types of membranes and the skin. Furthermore, candidate drugs possess a wide range of molecular size, shape, and chemical properties. Variations in the structure and chemistry of both the drug and the skin and mucous membranes contribute to the unpredictable nature of drug delivery. Furthermore, the costs of providing certain compounds that require separate studies for FDA approval can increase the costs of using purified or substantially pure compounds as permeation enhancers. In light of the recognized need to overcome the natural barrier properties of bodily membranes and skin in achieving drug bioavailability in an economical and prompt regulatory manner, the present invention relates to the provision of a mixture of class of compounds that enhance the permeation of drugs for delivery to a patient.

### SUMMARY OF THE INVENTION

In accordance with the present invention, there is provided a composition comprising a drug and a mixture of compounds which is effective in enhancing the bioavailability of said drug and which mixture comprises a major amount ofa compound having multi-carbon backbone having a functional group and also one or more side chains which have one or more carbon atoms and, optionally, one or more functional groups. A preferred class of mixtures of bioavailability-enhancing compounds comprises a major amount ofa compound of Formula I below.

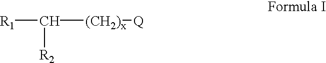

wherein,

x is 0 to about 18;

Q is

(1) a partially or completely neutralized —COOH, or

(2) a partially or completely neutralized —SO₃H, or

(3) a mono- or di-substituted alkyl or alkenyl group having one to about twelve carbon atoms, the substituent(s) thereof being a partially or completely neutralized —COOH or partially or completely neutralized —SO₃;

R₁ and R₂ are independently

(1) an unsubstituted alkyl or alkenyl group having one to about twelve carbon atoms, or

(2) a substituted alkyl or alkenyl group having one to about twelve carbon atoms, the substituent thereof being selected from the group consisting of

(i) partially or completely neutralized —COOH,

(ii) partially or completely neutralized —SO₃H,

(iii) —NH₂,

(iv) —CONH₂; and

(v) —OH; provided that the number of carbon atoms in R₁ and R₂, (CH₂)ₓ and Q is about 18 to about 22.

Another aspect of the present invention comprises a method of treating a condition in a patient comprising administering to the patient a composition comprising a pharmaceutically effective amount of a drug for treating the condition and a permeation enhancer of Formula I in an enhancing-effective amount.

As explained below, a particular advantage of the present invention is that it provides to the medical and pharmaceutical professions a class of compositions that, for drugs having widely different hydrophilic-hydrophobic properties, enhance the permeation of said drug into and through membranes, for example, the intestinal barrier of a subject and skin. These compositions comprise mixtures of compounds derived from various sources including natural sources and are typically low in cost yet effective in enhancing the delivery of drugs to a patient.

### DETAILED DESCRIPTION OF THE INVENTION

As mentioned above, the composition of the present invention comprises a drug, a compound mixture that is characterized herein as a permeation enhancer, and, optionally, a vehicle. Permeation enhancer compositions include a composition comprised of a mixture of compounds represented by Formula I. Consideration in the selection of the constituents of the composition is given to both the nature of the drug employed and to the tendency of the target membrane or skin to absorb the drug. A preferred source of the mixture of compounds from which permeation enhancer compositions are derived comprises preferably about 60 to about 95-weight % of compounds of formula I. A more preferred range is about 64 to about 80 weight percent. As will become evident from the following discussion, there is included within the class of enhancer compositions of the present invention mixtures including compounds that have a wide range of hydrophobic-hydrophilic properties and that may be described as branched chain compounds.

The compounds described in Formula I comprise a multi-carbon backbone having a functional group and also a side chain(s) which has one or more carbon atoms and, optionally, one or more functional groups. These compounds are therefore distinguished from the straight chain carboxylic acids reported in the literature as having permeation enhancer properties. Each of R₁ and R₂ of Formula I represents an unsubstituted alkyl or unsubstituted alkenyl group having 1 to about 12 carbon atoms or a substituted alkyl or substituted alkenyl group having 1 to about 12 carbon atoms, or one of R₁ or R₂ can be a substituted alkyl or substituted alkenyl group having 1 to about 12 carbon atoms and the other an unsubstituted alkyl or unsubstituted alkenyl group. Each of R₁ and R₂ of Formula I may be a straight or branched chain.

In addition, one of R₁ or R₂ can be an alkyl group and the other an alkenyl group. Examples of alkyl groups are methyl, ethyl, isopropyl, hexyl, octyl, decyl, and dodecyl. Preferably, the alkyl group has at least about 4 to about 12 carbon atoms. Examples of alkenyl groups are octenyl, pentenyl, and dodecenyl. Preferably, the alkenyl group has at least about 4 to about 12 carbon atoms.

Also, in preferred form, the sum of the carbon atoms in R₁ and R₂ and (CH₂)ₓ is at least about 18. In a particularly preferred form of the invention, R₁ is alkyl and R₂ is alkyl. For those enhancers in which R₁ and/or R₂ includes a substituted alkyl or substituted alkenyl group, it is preferred that the substituent thereof is a hydroxyl group.

As set forth in Formula I, enhancer compounds useful in the present invention can include a partially or completely neutralize Carboxylic acid (—COOH) or Sulforic acid (—SO₃ H) group. As used herein, the term “neutralized” means the reaction product of the carboxylic acid or sulfonic acid with a base that is present in an amount sufficient to react with all of the acid. As used herein, the term “partially neutralized” means the reaction product of the carboxylic or sulfonic acid with an amount of base that reacts with less than all of the acid, but with at least about 50 % of the acid. Examples of bases that can be used are sodium hydroxide, sodium carbonate, potassium hydroxide, magnesium hydroxide, calcium hydroxide, ammonium hydroxide, and trialkyl amine. Preferably, -Q of Formula I is the sodium salt of —COOH. For those enhancers where -Q of Formula I is a substituted alkyl or substituted alkenyl group, the following are examples of such groups: methyl, hexyl, octyl, and dodecyl. Preferably, the total number of carbon atoms in the alkyl or alkenyl group is about one to about 12, with an alkyl group being preferred.

In a preferred group of compounds of Formula I, R₁ is C₆-C₁₂ alkyl, R₂ is methyl, “x” is 3 to 8, and -Q is neutralized —COOH. Particularly preferred permeation enhancers are compounds represented by Formula I wherein R₁ is C₇-₉ alkyl, R₂ is methyl, x is 6 to 8 and -Q is —COONa.

A preferred enhancer composition useful in the present invention includes a mixture having a major amount of a compound that comprises the sodium salt of a carboxylic acid of Formula I in which the R₁, R₂ , and (CH₂)ₓ groups have a total of 17 to 20 carbon atoms, and most preferably a total of 18 carbon atoms. A natural source of the acids from which the enhancer compounds are derived, for example, EMERSOL 874®, can contain in addition about 6 to about 15 percent by weight of compounds which contain a total of about 18 to about 20 carbon atoms and have a structure according to Formula II, where the cyclohexane ring shown can be as well a cycloalkylene group of any size such that the total number of carbon atoms in structure II is about 18 to about 20, or of compounds according to Formula III where the aromatic group shown can be alkyl-substituted such that the total number of carbon atoms in structure III is about 18 to about 20 carbon atoms. “Cycloalkylene” means a saturated monocyclic hydrocarbon divalent radical. Preferred groups contain about 5 to about 12 carbon atoms, more preferably about 5 about 10-carbon atoms, even more preferably about 5 to about 7 carbon atoms. Examples of such cycloalkylene radicals include cyclopentylene, cyclohexylene, cycloheptylene, and the like.

Preferred compounds of Formula II and III including cycloalkylene or divalent aromatic groups, wherein x and y may be one to about 10, and are together from 10 to about 14.

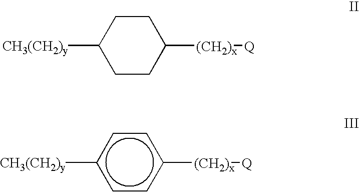

The enhancer compounds included in the mixtures useful in the present invention include at least one chiral center, and may be used as a racemic mixture of optical isomers, or optionally as the essentially pure D or L isomers.

Species of enhancer compounds within the scope of the present invention are known. Speaking generally, the enhancer carboxylic acids useful in the present invention can be prepared according to known preparative methods. Non-limiting examples of preparative methods include the oxidative cleavage of an appropriately unsaturated hydrocarbon with a strong oxidizing agent and the saponification of a corresponding ester. A non-limiting example of a typical ester is the glyceride of the desired acid.

Neutralization of a carboxylic acid or sulfonic acid with an alkali such as sodium hydroxide is generally carried out by adding the alkali to a stirred solution of the acid dissolved in water or a mixture of water and alcohol. The degree of neutralization is monitored by changes in pH as measured by conventional means.

The enhancer compound of Formula I can be mono-functional or multi-functional. The degree of functionality and length of the carbon chain are related to the hydrophilic-hydrophobic (lipophilic) nature of the enhancer compounds. In general, the higher the degree of functionality, the more hydrophilic is the compound. Also, speaking generally, the greater the number of carbon atoms in the compound, the more hydrophobic the compound is. Improved drug delivery can be achieved when the hydrophobic-hydrophilic balance of the enhancer is matched appropriately to the drug and to the targeted tissue. Selecting -R₁, -R₂ , x, y and -Q with relatively long carbon chains can provide enhancers having a relatively high degree of hydrophobicity. In contrast, enhancers with relatively short carbon chains and with multi-functional groups have a relatively high degree of hydrophilicity.

A most preferred enhancer composition comprises from at least 50% of a C18 branched chain carboxylic acid salt (a salt having a structure of formula II), from about 5 to about 15% of a C18 cyclic carboxylic acid salt, and from about 5 to about 15% of a C18 aromatic carboxylic acid salt. A most preferred commercially available material that may be used to prepare the composition according to the present invention contains about 68% of the C18 branched chain carboxylic acid, about 6% of the aromatic C18 carboxylic acid, and about 14% of the C18 cyclic carboxylic acid. This material is sold under the mark, EMERSOL 874®, as an isostearic acid by Cognis Corporation. The typical composition for EMERSOL 874® is found on the Cognis website, www.cognis-pmt.com, and is hereby incorporated by reference. This material may be completely or partially neutralized to yield a preferred enhancer composition.

The composition of the present invention comprises also a drug, for example, a chemical compound that has prophylactic, therapeutic, or diagnostic properties and which is used in the treatment of humans or other animals. The composition can comprise a mixture of two or more drugs.

It is believed that the present invention will be used most widely with drugs whose bioavailability and/or absorption properties can be enhanced by use of the permeation enhancer of the present invention. It is believed also that the present invention can be used to a particularly good effect by combining the permeation enhancer of the present invention with a drug that is ingested orally and absorbed relatively poorly in the gastrointestinal tract (“GIT”). Examples of such drugs are those that are known to have a relatively slow rate of membrane permeation such as, for example, Class III and Class IV drugs. Class III drugs are highly soluble in aqueous media with poor membrane permeability. Class IV drugs have low water solubility and low permeability.

Representative drugs in these classifications include, for example organic and inorganic therapeutic agents in the range of up to 400 daltons (the so called “small molecule” drugs) in proteins, peptides, vaccines, antigens, oligomers and polymers of ribonucleic acid (RNA) or deoxyribonucleic acid (DNA) or mimetics thereof including oligonucleotides and polynucleotides composed of naturally-occurring nucleobases, sugars and covalent inter-nucleoside (backbone) linkages as well as non-naturally-occurring portions which function similarly. Modified or substituted oligonucleotides and polynucleotides are often preferred over native forms because of desirable properties such as, for example, enhanced cellular uptake, enhanced affinity for nucleic acid targets and increased stability in the presence of nucleases. U.S. Pat. No. 6,379,960 teaches various suitable modifications and substitutions to oligonucleotides and polynucleotides.

Specific examples of drugs include “small molecule” drugs, for example furoseamide, low molecular weight (LMW) heparin, nucleotides, peptides and protein such as insulin, growth hormone, calcitonin, enalaprilate, acyclovir, leuprolide acetate, antisense oligonucleotides, ribozymes, external guide sequence (EGS) oligonucleotides (oligozymes), and short catalytic RNAs or catalytic oligonucleotides which hybridize to a target nucleic acid and modulate its expression. It will be appreciated that the aforementioned list of drugs includes examples of hydrophilic drugs and macro-molecular drugs.

The drug can be in any suitable form, for example, in crystalline or amorphous form and in solid, liquid, or gel form, for example, in the form of nano particles and micro particles or in larger particle-size form. In addition, the drug can be present in the composition in a time-release form.

The composition of the present invention comprises a pharmaceutically effective amount of the drug, that is, an amount that is effective in achieving the desired prophylactic, therapeutic or diagnostic effect in the patient. It should be appreciated that the amount of drug comprising the composition will depend on various factors, including, for example, the particular drug used, the nature of the condition to be treated, and the nature of the patient.

Similarly, the enhancer compound contained in the composition of the present invention is present in an amount that is effective in increasing the bioavailability and/or absorption properties of the drug. The amount of enhancer in the composition will depend on various factors, including, for example, the relative amount of each individual enhancer species present, the particular drug(s) used, the amount of drug(s) employed, the dosage form selected, the optical purity of the enhancer compound(s) used, that is, whether they are used in the form of a pure isomer or as a partially or completely racemic mixture. It is believed that, for most applications, the composition will comprise a drug: enhancer compound weight ratio of about 1:1000 to about 99:1. In most cases the ratio will be between about 1:5 and about 1:10. This ratio range is given for guideline purposes, with the understanding that ratios of drug to enhancer outside of this range may be used depending on the various factors mentioned above.

The composition of the present invention comprises optionally a vehicle, the nature of which will depend on the form of the composition. The composition can be used in any suitable form, for example, in the form of a tablet, a capsule and semi-solid. The tablets and capsules can be in the form, for example, of delayed release, sustained release, or immediate release systems. It is believed that the composition of the present invention will be used most widely in solid oral dosage form.

The term “vehicle” is used broadly to include various types of pharmaceutically acceptable ingredients that can comprise the composition other than the drug and enhancer constituents of the composition. Examples of vehicles include fillers, diluents, excipients and materials, which have an effect on the release properties of the drug, that is, control-release materials.

Examples of fillers and diluents include lactose, mannitol, dextrose, and microcrystalline cellulose.

Examples of excipients include phosphate and citrate salts, magnesium stearate, silica, and binders such as hydroxypropyl methylcellulose, polyvinylpyrrolidone, and starch. Examples of control-release materials include enteric polymers, hydroxypropyl methylcellulose.

The amount of the various classes of constituents that comprise the carrier can be selected by the user to achieve the desired effects.

The examples below are illustrative of the present invention and compare the present invention to prior art compositions.

### EXAMPLES

### Example 1

### LMW Heparin Composition including EMERSOL 874®

An enhancer composition was prepared by completely neutralizing 100 g of EMERSOL 874® in 50 ml of warm water with 40 ml of isopropanol added as a co-solvent. Aliquots of a 20% sodium hydroxide aqueous solutions were added until a pH=7 was obtained in the solution. The solvent was evaporated and the solid acid salts thus obtained were used as prepared.

The performance characteristics of the mixture of carboxylic acid salts, prepared from EMERSOL 874® as described above, containing about 68% of the sodium salt of a branched chain C18 carboxylic acid, is compared with the performance of the straight chain sodium carboxylic acid, the sodium salt of capric acid, in a study of the intestinal absorption of LMW heparin (parnaparin) when administered by intra-duodenal cannula to the conscious rat model.

The comparison is carried out in a non-randomized, parallel group design, and the animals used are male Wistar rats (25) in the 250-350 g-weight range (n=7 for each formulation). Animals are surgically implanted while under anesthesia with a duodenal cannula and a venous (jugular vein) catheter for formulation administration and blood sampling respectively. The rats are allowed to recover for at least one day prior to dose administration. LMW heparin (Fluxum parnaparin-mean molecular weight 4000-4500 Dalton) formulations as described below are prepared in a phosphate buffer saline (0.01 M, pH 7.4) and are administered as a bolus (0.3 ml) into the duodenum. Blood samples are taken from the jugular vein at the following time intervals: 0 (pre-dose) 5, 10, 15, 30, 45, 60, 120, 180, 240 and 360 minutes. The samples are collected into epindorfs containing trisodium citrates and plasma is separated by centrifugation at 3000 rpm for 15 minutes. Plasma samples are stored at −20° C. until analysis. Samples are analyzed using Chromogenix Coatest® Heparin Kit and results expressed as antifactor Xa activity (IU/ml). The relative bioavailability (i.e. relative to a subcutaneous does of heparin 250 IU per animal) is calculated from the areas under the curve obtained from plasma antifactor Xa concentration-time profiles:

The formulations administered to subjects in the comparison study are given in Table 1, below.

TABLE 1

| Group | |
|---|---|
| No. | Treatments |
| A | 1000 IU LMWH (Parnaparin) + 35 mg Enhancer (ID) |
| B* | 1000 IU LMWH (Parnaparin) (ID) |
| C* | 1000 IU LMWH (Parnaparin) = 35 mg C10 (ID) |

In the chart above, ID is intraduodenal, enhancer (1) is EMERSOL 874, and C10 (2) is the sodium salt of capric acid.

The pharmacokinetic measurements (mean±SD) obtained are presented in Table II, below.

TABLE 2

| | Treatments | | |
|---|---|---|---|
| | Treatment A | | |
| | 1000 IU LMWH | | |
| | (Parnaparin) + | | Treatment C |
| | 35 mg | Treatment B | 1000 IU LMWH |
| | Enhancer + | 1000 IU LMWH | (Parnaparin) + |
| PK | 0 mg C10 | (Parnaparin) | 0 mg Enhancer + |
| Parameters | (ID) | (ID) | 35 mg C10 (ID) |
| % Fᵣₑₗ | 3.37 ± 3.84 | 0.37 ± 0.66 | 3.06 ± 3.14 |
| AUC | 2.49 ± 2.84 | 0.26 ± 0.47 | 2.16 ± 2.22 |
| (IU/ml.h) | | | |
| Cmax | 1.94 ± 2.33 | 0.30 ± 0.38 | 1.61 ± 1.37 |
| (IU/ml) | | | |

All above groups are dosed intra-duodenally (ID); % Fᵣₑₗ = % relative bioavailability.

In the conscious rat model, the bioavailability of LMW heparin dosed to animals without any permeation enhancers is very low (less than 0.5%). This however, significantly improved when the drug dosed is combined with a permeation enhancer. The highest bioavailability is observed when heparin is dosed with the permeation enhancer derived from EMERSOL 874. The enhancement of bioavailability with this branched chain compound mixture is slightly greater that that achieved with the straight chain carboxylic acid, sodium caprate.

More specifically, the relative bioavailability following the administration of 1000IU parnaparin (ID) is 0.37±0.66%. When 10000IU parnaparin is co-administered with 35 mg C10 (sodium caprate), the resultant relative bioavailability is 3.06±3.14%. The highest relative bioavailability observed follows the administration of 1000IU parnaparin+35 mg branched chain enhancer mixture i.e. 3.37±3.84%.

From the above description, it should be appreciated that the present invention provides a method of drug delivery which overcomes the natural barrier properties of bodily membranes and skin in such a way that bioavailability of the drug is improved significantly and pharmaceutically effective amounts of drugs can be provided at a sustainable rate over an extended period of time. Furthermore, the permeation enhancer used comprises a relatively inexpensive and generally recognized as safe (GRAS) approved material that is capable of accelerating the drug development process.

Although enhancers of the present invention are useful in applications involving drug delivery across the skin and various mucous and other cellular membranes, they are especially effective in improving the bioavailability of drugs that are ingested orally and then absorbed in the GI tract.

While not wishing to be bound by a scientific theory regarding the mechanism by which the drug delivery system of the present invention functions, it is believed that the drug is transported through the skin or membrane barrier by the chemical processes of diffusion and capillary action. For example, the resistance or barrier property of the skin or membrane is due at least in part to the highly ordered intercellular lipid structure of the stratum corneum, a phospholipid bilayer membrane. The permeation enhancer may disrupt and reduce the orderly structure of the stratum corneum, thus making the cell structure more fluid. This allows higher rates of drug permeation by diffusion. Concurrently with increased diffusion rates (as result of disruption of the stratum corneum), the permeation enhancer causes an increase in the surface activity of the drug molecule itself, thus effecting a faster movement of the drug through the skin structure.

Drug permeation rates are influenced by factors related both to the membrane and to the drug itself. With respect to the membranes, the individual cellular units are a major factor in controlling the permeation rate of a drug. The plasma layer surrounding each cell is comprised of phospholipids having alternating hydrophilic and hydrophobic layers which serve a protective function, but which also pose a barrier to many drugs. The nature of this barrier may vary among the membranes of the body. Drugs generally vary in chemical properties such as solubility, polarity, and molecular size and, therefore, have variable rates of diffusion through bodily membranes. Because each combination of drug and target membrane within the body presents a unique environment for permeation, the pathways to achieving adequate bioavailability levels are typically complex and unpredictable. It is believed that the enhancers of the present invention provide an improved solution to the problem of effective permeation by enabling one to use relatively inexpensive and GRAS approved mixtures that optimize the formulation of compositions, which are particularly effective for delivering drugs

We claim:

1. A pharmaceutical composition comprising a drug and a permeation enhancer that comprises a mixture of compounds, said mixture containing a major amount of compound having a multi-carbon backbone having a functional group and also one or more side chains which have one or more carbon atoms and, optionally, one or more functional groups.

2. A composition comprising:
(a) a drug,
(b) a mixture of compounds containing a major amount ofa compound of Formula I: 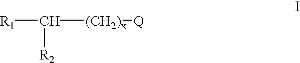
wherein:
x is 0 to about 18;
Q is
(1) a partially or completely neutralized COOH, or
(2) a partially or completely neutralized SO₃H, or
(3) a mono or di-substituted alkyl or alkenyl group having one to about 12 carbon atoms, the substituent(s) thereof being a partially or completely neutralized —COOH or —SO₃H;
R₁ and R₂ are independently
(1) an unsubstituted alkyl or alkenyl group having one to about 12 carbon atoms.
(2) a substituted alkyl or alkenyl group having one to about 12 carbon atoms, the substituent thereof being selected from the group consisting of a neutralized or partially neutralized —COOH or —SO₃H, —NH₂, —CONH₂; —OH; provided that the number of carbon atoms in R₁ and R₂, (CH₂)ₓ and Q is about 18 to about 22, and
(c) optionally, a pharmaceutically acceptable vehicle.

3. A composition according to claim 2 wherein said compound of Formula I is a compound wherein:
Q is —COONa,
x is 1,
R₁ is —C₁₄ straight chain alkyl, and
R₂ is —methyl.

4. A composition according to claim 2 wherein said compound of Formula I is a compound wherein:
Q is —COONa
x is 2,
R₁ is —C₁₃ straight chain alkyl, and
R₂ is —methyl.

5. A composition according to claim 2 wherein said compound of Formula I is a compound wherein:
Q is —COONa
x is 3,
R₁ is —C₁₂ straight chain alkyl, and
R₂ is —methyl.

6. A composition according to claim 2 wherein said compound of Formula I is a compound wherein:
Q is —COONa
x is 4,
R₁ is —C₁₁ straight chain alkyl, and
R₂ is —methyl.

7. A composition according to claim 3 to 6 including a minor amount of a compound of Formula I wherein:
Q is —COONa;
wherein the total number of carbon atoms is about 18 to about 20 and R₁ and R₂ form a cycloalkyl group or an aromatic group.

8. A method of treating a condition in a patient comprising administering to the patient a composition according to claim 1 containing said drug in a pharmaceutically effective amount and said a mixture of compounds containing a major amount ofa compound of Formula I in a permeation enhancing-effective amount.

9. A method according to claim 8 wherein Q is —COONa, R₁ is —C₁₂ straight chain alkyl, and R₂ is —C₅ straight chain alkyl.

10. A method according to claim 8 wherein Q is —COONa, R₁ is —C₁₁ straight chain alkyl, and R₂ is —C₆ straight chain alkyl.

11. A method according to claim 8 wherein Q is —COONa, R₁ is —C₁₀ straight chain alkyl, and R₂ is —C₇ straight chain alkyl.

12. A method according to claim 8 wherein Q is —COONa, R₁ is —C₉ straight chain alkyl, and R₂ is —C₈ straight chain alkyl.

13. A method according to claim 8 including a minor amount of a compound of Formula I wherein:
Q is —COONa; and
wherein the total number of carbon atoms is about 18 to about 20 and R₁ and R₂ form a cycloalkyl group or an aromatic group.
